(19) United States
(12) Patent Application Publication
(10) Pub. No.: US 2010/0304466 A1
(43) Pub. Date: Dec. 2, 2010

(54) System for Supplying an Automatic Inoculating Device and Process Using Such an Automatic Device

(75) Inventor: Emmanuel JALENQUES, St Nom la Breteche (FR)

Correspondence Address: THOMAS P O'CONNELL, 1026A MASSACHUSETTS AVENUE, ARLINGTON, MA, 02476, US

(73) Assignee: INTERLAB, St Nom la Breteche (FR)

(21) Appl. No.: 12/791,044

(22) Filed: Jun. 1, 2010

(30) Foreign Application Priority Data
Jun. 2, 2009 (FR) 09 02644

(51) Int. Cl. C12M 1/26; C12N 1/00

(52) U.S. Cl. 435/243; 435/309.1

(57) ABSTRACT

A supplying system for an automatic inoculating machine for a sample on a substrate, characterized in that it includes a turret mobile in rotation, preferably around an axis substantially vertical, and an arm mobile in rotation, preferably around an axis substantially horizontal, carried by the turret, at a distal end of the aforesaid arm including means to fix a stylus, and means for raising or lowering the arm.

### FIELD OF THE INVENTION

The present invention relates to the field of automatic devices for inoculating a culture substrate with a sample to be analyzed, usually a substantially liquid sample. It relates more particularly to a system to take and inoculate the sample, for example on a substrate in a Petri dish.

### BACKGROUND OF THE INVENTION

In an automatic inoculating device, each sample is taken in a sample tank by the automatic device using a stylus, and then distributed on the substrate surface using the same stylus. Between each inoculation, it is proper to clean the stylus to avoid contamination of the next sample . . . .

In the automatic devices of the prior art, stylus is dipped in several tanks, the first one containing a disinfectant, the following two containing sterile water for rinsing the disinfectant. Such a method is described in the document U.S. Pat. No. 5,547,872 o Schalkowsky et al.

The stylus must be moved from one area of sample collection in the corresponding tank to an inoculating area in which the sample must be distributed, generally in a spiral pattern, then to each cleaning station and then into each cleaning tank. In all cases, the moving of the stylus is linearly, for example through a drive rack.

At each position, the stylus must be lowered or raised, for example, to dip into the sample tank or in a tank of cleaning fluid.

The maintenance of the devices of the prior art is delicate. In addition, they do not have a satisfactory reliability for the realization of the pattern of the inoculation.

The invention aims to provide a system that can answer to the previously mentioned drawbacks, particularly a simple and inexpensive system, allowing for easy, reliable and efficient handling of the sample and possibly of the cleaning products.

### SUMMARY OF THE INVENTION

According to the invention, such a supplying system of an automatic inoculating device for a sample on a substrate is characterized in that it comprises a mobile turret in rotation, preferably around a substantially vertical axis, and an arm mobile in rotation, preferably around a substantially horizontal axis, carried by the aforesaid turret, a distal end of the aforesaid arm including means to fix a stylus, and means for raising or lowering the aforesaid arm. The means for raising or lowering the aforesaid arm includes a movable piston, from bottom to top and vice versa, the aforesaid piston being arranged, preferably near the axis, so that the arm is resting on an upper end of the aforesaid piston.

The means for fixing the stylus are preferably fixing means without tools. They may advantageously comprise:
a ring intended to be fixed, preferably glued, on the stylus;
means of axial and transverse positioning of the ring relative to the distal arm, and
means of keeping the ring on the distal arm.

The axial and transverse positioning means may include a conical surface on the ring, and a conical surface of housing formed in the distal end, the aforesaid conical surfaces having substantially the same angle, and preferably expected to trap one in the other. The means of holding in place the ring may include a peripheral rib on the ring, and a nut provided for screwing on the cap and cooperating with the rib to keep the ring gripping with the extremity.

The stylus is advantageously formed in one extremity of a flexible pipe. Preferably, this pipe is fixed so that it is fully accessible and mountable and/or removable without tools. To ensure that only the pipe may be contaminated by the sample, the pipe has preferably a length so that it provides an internal volume sufficient to store a sample volume sufficient for the inoculation. In order that this length is properly disposed in the controller, it may include a roll, to wind around a portion of the pipe.

In addition, the system of the invention advantageously comprises motorized pumping means, preferably with a syringe, placed upstream of the pipe, provided for at least sucking up and distributing the sample by the stylus. It may also include dispensing means settled so that the pumping means can also suck up at least one product in a relevant tank, and then reject that aforesaid product by the stylus.

Preferably, the means of distribution are settled so that the pumping means can draw two products, independently of one another, each product having a specific reserve, and discharge independently each product by the stylus. Advantageously, one product is a disinfectant, preferably an alcohol; the other is rinse aid, preferably distilled water.

The system advantageously comprises means to adapt a Petri dish expected to contain the substrate.

The invention also relates to a method of inoculating a Petri dish using a system according to the invention.

### BRIEF DESCRIPTION OF THE DRAWINGS

Several embodiments of the invention will be described below, as no limiting examples, with reference to the accompanying drawings in which:

FIG. 1 illustrates schematically the operation of an inoculating automatic device according to the invention;

FIG. 2 is a section which illustrates schematically a system according to the invention for the supply of the automatic device of FIG. 1, the system comprising an arm mounted on a rotating turret;

FIG. 3 is a half-section illustrating means for fastening a stylus to the extremity of the arm of FIG. 2;

FIGS. 4 and 5 illustrate different modes of implementation of a process of inoculating according to the invention;

FIG. 6 is a sectional view of storage and sampling area for a product to be inoculated in the automatic device of FIG. 1;

FIG. 7 is a sectional view of means for cleaning the stylus of FIG. 3;

FIG. 8 is a view similar to FIGS. 4 and 5, in which the Petri dish is square and the inoculating is made in the form of straight lines parallel to each other; and,

FIG. 9 is a view similar to FIG. 8, where inoculation takes the form of squares substantially homothetic.

### DETAILED DESCRIPTION OF PREFERRED EMBODIMENTS

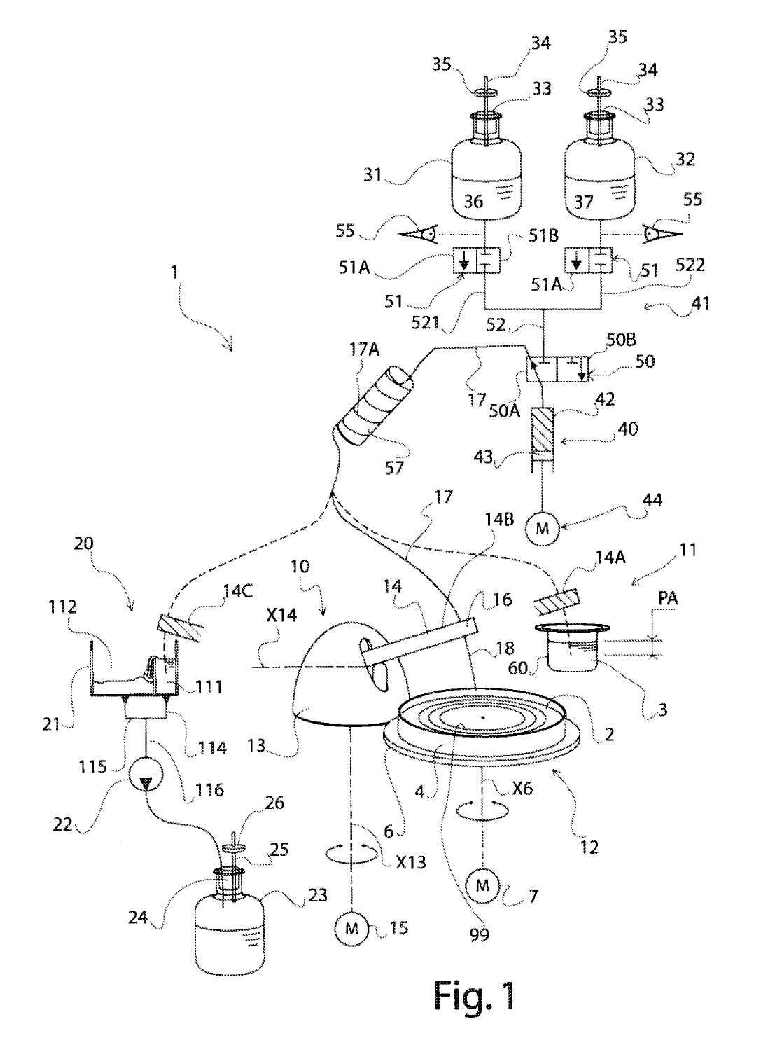

FIG. 1 illustrates a system of automatic system to inoculate a substrate 2 with a sample to be analyzed 3. In the illustrated example, the substrate is contained in a Petri dish 4, under the shape of a gel, and the product 3 is substantially liquid.

The illustrated system includes a supply area 11 for the sample to be tested and an inoculating area 12. It includes means 10 to collect the sample 3 in the supply area and deposit it, at least partially, on the surface of the substrate 2. The automatic device includes a tray 6, turning around a vertical axis X6. The tray 6 constitutes a support for the Petri dish 4. It is at least indirectly rotated around its axis X6 by a motor 7.

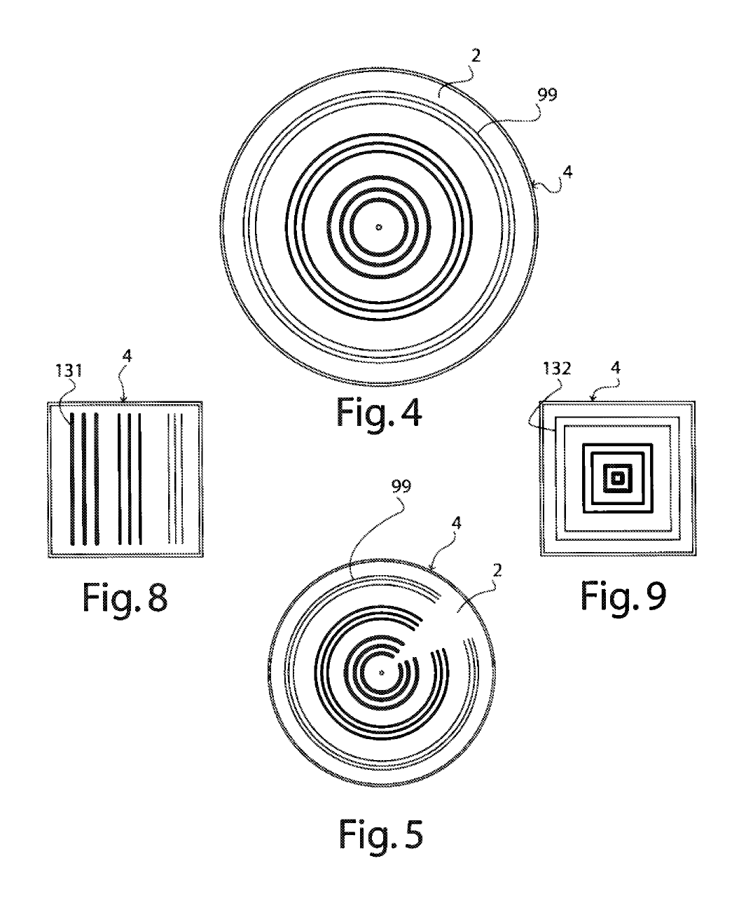

A method of inoculating according to the invention is more particularly described with reference to FIGS. 4 and 5.

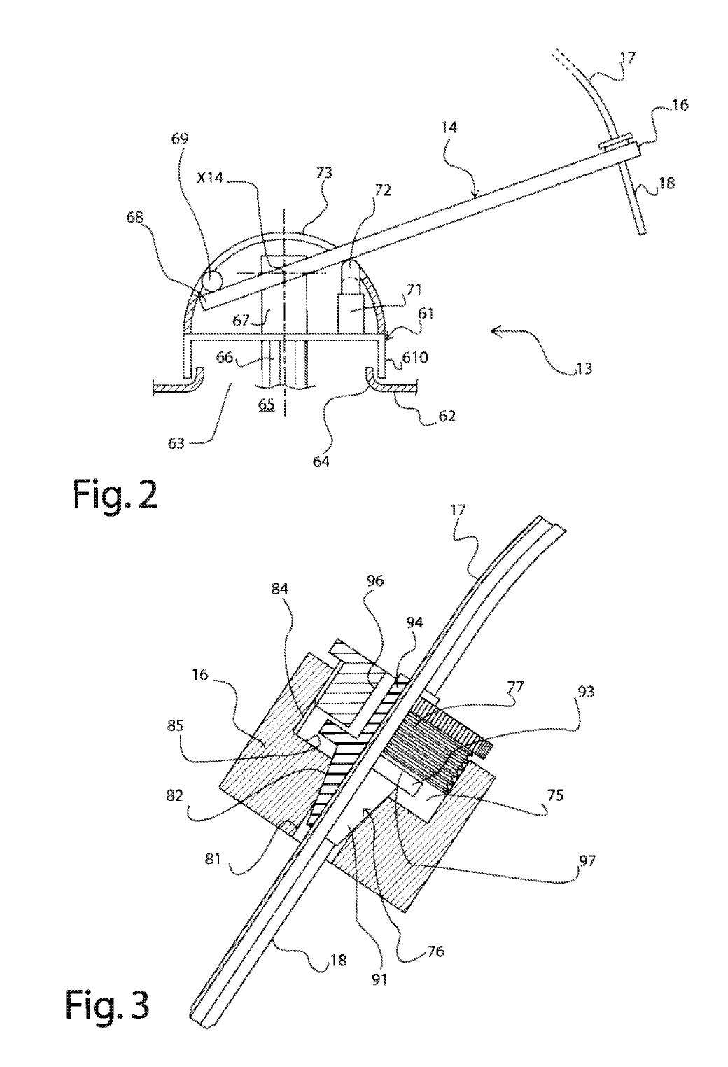

The taking means include a turret 13, mobile in rotation around a vertical axis X13, under the action of a motor 15. The turret is equipped with an arm 14. The arm 14 is movable in a vertical plane, relative to the turret 13, rotating around a substantially horizontal axis X14 carried by the turret 13. A distal extremity 16 of the arm 14 carries a flexible pipe 17 with one extremity 18 of the aforesaid pipe, extending downwards from the arm 16 forms a stylus 18. The turret will be described in more details with reference to FIGS. 2 and 3. Preferably, the pipe is made of a material not adhering, for example POLY (TETRAFLUOROETHYLENE). This arrangement is particularly advantageous in that it limits the adhesions, especially when a sample has a relatively thick and sticky consistency.

The automatic device of FIG. 1 includes further cleaning means 20 for the stylus. In the illustrated example, the cleaning means 20 include a pour tank 21, draining means 22 for discharging effluent from the cleaning tank 21 and retention vessel 23 for the aforesaid discharged effluents by pumping. In the example shown, the draining means 22 include a diaphragm pump. The retention vessel is closed by a plug 24. The plug is pierced by a vent 25, having the shape of a pipe. This pipe 25 is equipped with a filter 26 of 0.2 μm, so the atmosphere is protected from any possible microbial contamination. The pour tank 21 and its use will be more particularly described with reference to FIG. 7.

The automatic device of FIG. 1 also includes reserves 31 and 32 for containing liquid for cleaning the stylus 18. Preferably each of these reserves is removable and can be replaced by a full one, whenever necessary. Each reserve 31.32 is shaped like a bottle closed by a plug 33 with a vent 34 for maintaining the interior of the cylinder at atmospheric pressure as to the transfer of liquid it contains.

Each vent 34 is fitted with a filter 35, e.g. a filter 0.2 μm, to ensure the sterility of the liquid contained in the corresponding reserve. An initial reserve 31 of the two reserves contains a disinfectant 36, alcohol 36 in the example shown. The second reserve 32 contains rinse aid 37, distilled water 37 in the example shown.

The automatic device includes further pumping means 40 and supplying means 41 for different fluids 3, 36, 37 manipulated by the automatic device 1. In the example illustrated, the pumping means includes a syringe 40 engineered by a cylinder 42 in which slides a piston 43. The piston is driven by a motor 44, preferably a stepper motor.

The supplying means are here schematized by three valves 50, 51. A first valve 50 among these three valves includes two positions; the first position, shown in the figure, can suck up or reject a fluid through the pipe 17. The second position of the first valve 50 allows connecting the syringe 40 with a supplying pipe 52 for cleaning fluid 36, 37. The pipe 52 comprises two parts 521 and 522, each engaging with a respective reserve 31.32 of cleaning fluid.

Each second and third valve 51 is assigned to a respective reserve of 31.32 cleaning fluid 36, 37. In a first opened position 51A each valve 51 allows the fluid to flow in the respective pipe 52. In a second closed position 51B, shown in FIG. 1, each valve 51 prevents the respective liquid to flow out in the pipe 52.

A sensor 55 is arranged on each side piece 521.522. The sensor 55 is provided to detect the absence or presence of liquid in the side piece. The absence of liquid in one of the side pieces commands the stopping of the automatic device 1 and the replacement or filling the corresponding reserve.

It is preferable that the sample taken from the pipe is maintained sufficiently downstream of the syringe 40, so that it cannot be contaminated by the sample.

Thus, the pipe is provided with a sufficient length so that its internal volume may contain a sufficient sample for inoculating. To enable the arrangement of this length of the pipe inside of the automatic device 1, it includes a roll 57 round which is wound a portion 17A of pipe 17. Preferably, the roll 57 includes a shaped screw along which the pipe portion 17A is arranged, and preferably fixed by snap inside the screw.

Thus prepared, the pipe (17) is visible and accessible throughout its length. It is assembled by interlocking and snap, so it is removable without tools.

In the example shown, the automatic device includes a stainless steel body, on which are arranged the various elements that compose it. The body is not shown in FIG. 1.

The body includes a substantially horizontal platform 62, particularly visible in FIGS. 2, 6, 7 and 8.

We will now describe generally a cycle of inoculating.

A sample is first stored in the supply area, for example in a container 60.

The arm 14 is brought in a sampling position 14A so that the stylus 18 is above the container 60. The arm 14 is then lowered so that the stylus dips to a PA depth into the sample. The valve 50 being in the position 50A, a sufficient portion of the sample is sucked up into the pipe using the syringe 40.

The arm is then raised, and then brought into a position 14B by rotating the turret 13 around its axis X13, particularly illustrated in FIG. 1, for the inoculation of the substrate 2. The arm 14 is lowered again so that the stylus is close enough to the substrate to deposit the sample with the desired precision. By a combination of rotating movements of the turret 13 around its axis X13 and the platform 6 around its axis X6, the sample is automatically deposited according to a previously defined pattern. The pattern may be a spiral or a combination of points and/or circles or arcs of concentric circles, as illustrated with reference to FIGS. 4 and 5.

Once inoculating is completed, the arm is raised, and then taken to a cleaning position 14C by rotation of the turret 13 around its axis X13, to clean the stylus. The remaining sample still in the pipe is expelled into the discharge tank 21, using the syringe 40; the first valve 50 is always in the position 50A. Then the arm 14 is lowered so that the stylus 18 dips in the discharge tank.

The valve 50 is placed in the position 50 B, the third valve 51 is kept closed, in position 51B, the second valve 51 is placed in the position 51A, and the syringe is operated so that it fills with alcohol 36. Then, the positions of the first valve 50 and the second valve 51 are reversed, and the piston 43 is pushed inside the cylinder 42, so that the alcohol 36 is expelled into the tank 21, by browsing the entire length of the pipe 17. The inside of the pipe 17 is thus fully disinfected. The exterior of the pipe at the location of the stylus is disinfected since the tank 21 is filled with alcohol.

The valve 50 is returned to the position 50 B, the second valve 51 is kept closed, in position 51B, the third valve 51 is placed in the position 51A, and the syringe is operated so that it fills with distilled water 37. Then, the positions of the first valve 50 and the third valve 51 are reversed, and the piston 43 is pushed inside the cylinder 42, so that distilled water is expelled into the tank 21, by browsing the entire length of the pipe 17. The inside of the pipe 17 is thoroughly rinsed. The exterior of the pipe at the location of the stylus is flushed since the tank 21 is filled with distilled water 37.

A new cycle can then be started.

The sample is maintained downstream of the syringe; this syringe and the upstream portion of the pipe contain alternatively only alcohol 36 or water 37. This is that residual water that serves as a liquid piston between the piston 43 of syringe 40 and the sample, when the sample is handled, first to take and then to inoculate it.

We will now describe the turret 13 in reference to FIG. 2. The turret base 61 includes a substantially disc-shaped and equipped with a peripheral skirt 610. The platform 62 includes a circular orifice 63. A raised edge 64 is formed in the platform 62 in the periphery of the orifice 63. The skirt 610 is provided for covering the raised edge 64, so that they contribute together to prevent the penetration of liquid and/or of solid inside the body 65 of the automatic device 1.

Drive means 66 extends below the base to inside the body. They are fastened, at least indirectly, to the motor 15 of the turret 13. The base also carries a clevis 67 that defines the fail over horizontal axis X14 of the arm 14 and bearing the aforesaid arm. Opposite to its distal extremity 16, relative to the axis X14, the arm includes a proximal extremity 68 on which is fixed a counterweight 69, so that the fail over of the arm is substantially without effort. The proximal extremity 68 and counterweight 69 are directly above the base 61.

A cylinder 71 extends vertically upward from the base 61. The cylinder is placed close to the clevis 67, between the clevis and the distal extremity 16. The arm rests by its own weight on the upper extremity 72, moving vertically, of the cylinder 71. Thus, the arm 14 is movable in a vertical plane carried by the turret 13. The distal extremity 16 of arm 14 rises or falls with the extremity 72 of the cylinder 71. A hemispherical cap 73 covers and protects the inside of the turret 13.

FIG. 3 describes a particular arrangement for fastening the pipe to the extremity 16 of the arm 14. This assembly includes the following elements, each substantially for revolution, and mounted coaxially with each other:
a housing 75 running through the extremity 16 from top to bottom;
a ring 76 intended to be glued on the pipe, the part of the pipe exceeding downstream the ring is forming the stylus 18;
a nut 77 for maintaining the ring in the housing

The housing 75 includes, from bottom to top, a cylindrical portion 91 of small diameter, sufficient for the stylus 18 to be insert through, then a conical widening portion 82 and a cylindrical portion of large diameter 84 forming with the conical portion an escarpment 85. The interior portion 85 is threaded.

The ring 76 comprises, upstream to downstream, a conical portion 91 gradually widening at an angle identical to the portion 82 of the housing 75. The conical portions are provided to cooperate each other to position transversely and longitudinally the ring in the housing 75, likewise the stylus relatively to the extremity 16 of arm 14. The largest diameter of the portion 91 is greater than the largest diameter of the portion 82, so that the portion 91 extends beyond the portion 82, inside the cylindrical portion 94. Beyond the conical portion 91, the ring includes an annular rib 93 extending radially beyond the conical portion, and a cylindrical portion 94, radially away from the rib 93.

The nut 77 includes an axial cylindrical drilling 96, provided for the passage of the cylindrical portion 94 of the ring 76 and an anterior face 97 scheduled to come to bear on the rib 93 of the ring. Thus, when the assembly is achieved, the nut being in tune with the tapping of the housing 75, the anterior surface of the nut comes to bear against the rib and keeps the ring in position in the housing. Thus, the stylus is then maintained in a fixed and defined position relative to the extremity 16 of the arm.

We will now describe the reasons for improved inoculating a Petri dish 4.

In the automatic device of the prior art, the sample is inoculated in a pattern forming a spiral on the substrate. The pattern is achieved by moving radially the stylus at a constant linear speed, while the Petri dish turns on itself to constant angular velocity. This method is particularly advantageous in that it allows to gradually reducing the surface density in sample as it goes away from the center of the Petri dish. However, interpretation of results is complicated and requires special charts specific to the used inoculating device. The risks of misinterpretation are important.

It is proposed according to the invention to produce patterns as concentric circles 99. The density varies with the distance from the center of the Petri dish, as the spirals of the prior art, but it remains constant on a same circle, the interpretation is simplified because it depends no longer on the subtended angle under which the result of the culture is analyzed. In the example shown in FIG. 4, the pattern includes three groups of three close circles together. The circles of each group are very close; they have a density very close. Thus, each group corresponds noticeably to a determined concentration. To improve the accuracy of results, the automatic device advantageously comprises means for varying the speed of rotation of the Petri dish so that the density is substantially identical to the circles of the same group.

In the operating mode of FIG. 5, shown on a reduced scale, circles have been confined to arcs of circle 99. This pattern produced similar results. However, it avoids depositing the sample on previously inoculated area when achieving the circle.

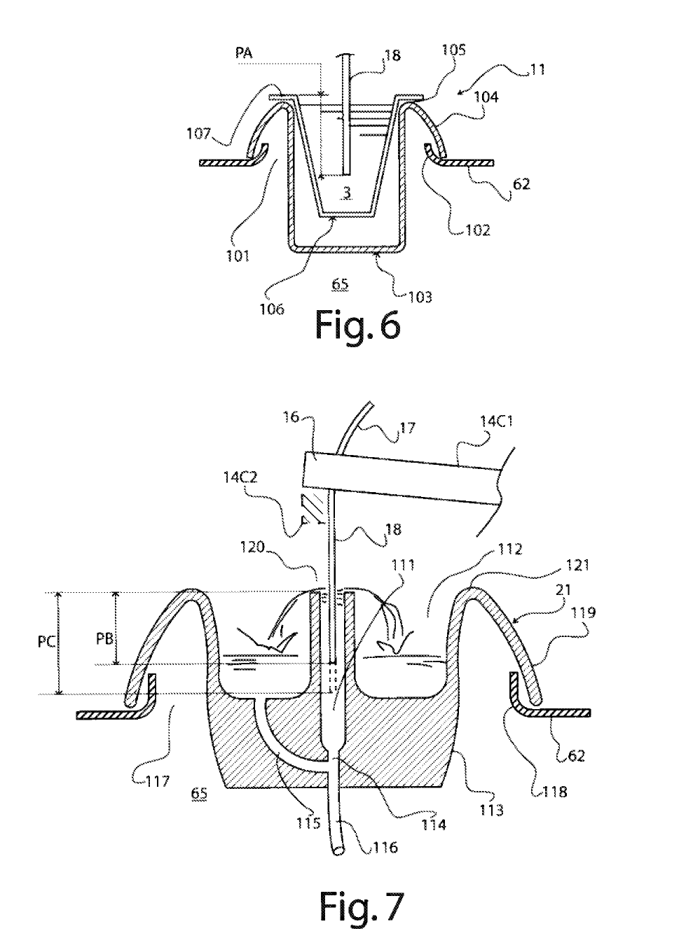

We will now describe the sampling area 11, with reference to the section of FIG. 6. Area 11 includes a circular orifice 101 in platform 62; the peripheral edge of the hole is shaped like a raised edge 102. A cylindrical tank 103 is disposed in the orifice 101. A skirt 104 extends from the upper edge 105 of the container and comes to rest on the platform 62, around the aforesaid raised edge 102. The skirt 104 covered the aforesaid raised edge 102, so that they contribute together to prevent the penetration of liquid and/or of solid inside the body 65 of the automatic device 1.

The product to be inoculated, that is to say the sample 3 is contained in a cup 106 whose upper edge 107 rests on the upper edge 105 of the container 103. Thus, a sample 3 may be supplied or removed from the automatic device without the risk of spilling in the body of the device that remains protected by the container 103. In addition, if the product 3 is spilled in the container, it is removable and can be removed for cleaning.

During the taking of the sample, the stylus 18 is expected to dip of a depth PA, measured at the edge 107 of the cup 106.

We will now describe the operation of the pouring tank and the cleaning process, referring to FIG. 7.

The pour tank 21 has substantially a shape of revolution around a vertical axis. It includes two coaxial bowls 111, 112, having a common base 113. The interior bowl 111 is designed specifically to receive the stylus 18 and cleaning fluids 36, 37. Its shape is narrow, so it offers radially sufficient, but without excess, space to provide the stylus and allow a flow of liquid 36.37 around the stylus. The outer bowl 112 is designed to recover the liquid flowing inside the bowl 111 when it overflows. Both bowls 111,112 include drain pipes respectively 114.115, formed in the base 113, and which join here to form only one 116, connected to the emptying pump 22.

As the turret 13 and the container 103, the discharge tank 21 is inserted into an orifice 117 of the platform 62. The tank 21 includes a skirt 119 which extends from an upper edge 121 of the outer bowl 112 and covers a raised edge 118 of the orifice 117. The device, as explained above, for the container 103 protects the interior 65 and can make the tank 21 easily removable, especially for cleaning.

When inoculating the substrate 2 is completed, as previously explained, the extremity of the arm 14 is rotated to a position 14C in which the stylus 18 is above the tank 21, preferably above the outer bowl 112, so that the pipe 17 is purged from the sample remaining not used for inoculating.

Then the arm is moved at position 14C1, in which the stylus 18 is dipped at a depth of PB into the interior bowl 111. The stylus is maintained at this position during the disinfection operation. As previously described, during this operation, alcohol 36 flows into the pipe so it drains out of the stylus 18. Alcohol then fills the internal bowl above its upper rim 120 since it overflows inside the outer bowl. Thus, the level of alcohol in the interior bowl 111 is always the same, substantially equal to the upper rim 120 of the bowl 111. The depth PB is chosen higher than the PA taking depth of the sample in the bowl 106. This step ensures the external disinfection of the stylus 18, on any height that may have been contaminated during the taking of the sample.

Then the arm is moved into position 14C2, in which the stylus 18 is dip at a depth PC into the interior bowl 111. The stylus is maintained in this position during the flushing operation. As previously described, during this operation, water 37 flows through the pipe to drain out off the stylus 18. The water then fills the internal bowl since it overflows the outer bowl, above its upper rim 120. Thus, the water level inside the bowl 111 is always the same, substantially equal to the upper rim 120 of the bowl 111. The depth PC is chosen higher than depth PB previously used for disinfection. This process ensures that alcohol previously used for cleaning out the stylus 18 is thoroughly rinsed and will not accidentally sterilized future inoculating.

FIGS. 8 and 9 illustrate two modes of implementation for an inoculating method according to the invention. In these examples, the Petri dishes 4 are square.

In the example shown in FIG. 8, the inoculation is done in a form of straight lines 131 parallel to each other, having substantially the same length. Lines 131 are grouped into three groups of three lines. The lines of the same group have a density approximately the same between themselves. The group represented on the left includes three lines of high density, the group represented on the right has three lines of low density, and the middle group includes three lines of intermediate density.

In the example shown in FIG. 9, the inoculation is done in a form of straight lines grouped in square 132. The squares are homothetic themselves around the same center. The squares 132 are grouped into three groups of two squares. The lines of all the squares of the same group have a density approximately the same between themselves. The most inside represented group include lines with high density, the most outside group includes lines with low density, and the intermediate group includes intermediate-density lines.

Of course, the invention is not limited to the previous described examples.

Thus, rather than a turret, it may be provided means of linear displacement for the stylus.

Also, instead of being provided removable, the pouring tank or the container of the sample zone may be provided fixed and shaped by stamping directly into the platform of the automatic device.

The pouring tank, as schematically illustrated in FIG. 1, may have a rectangular shape, and consist of two compartments separated by a wall for the pouring from one compartment to another.

Instead of only circular patterns, it can besides be planned particular patterns with in particular points more or less thick.

1. A system for supplying an automatic inoculating device for a sample on a substrate, the system comprising a turret mobile in rotation, preferably around an axis substantially vertical, and an arm mobile in rotation, preferably around an axis substantially horizontal, carried by the aforesaid turret, a distal end of the aforesaid arm including means to fix a stylus and means for raising or lowering the aforesaid arm.

2. The system according to claim 1 wherein the means for raising or lowering the aforesaid arm includes a piston moving from bottom to top and vice versa, the aforesaid piston being arranged, preferably near the axis, so that the arm is resting on an upper end of the aforesaid piston.

3. The system according to claim 1 wherein in that the means to fix the stylus are ways of fixing without tools.

4. The system according to claim 1 wherein the means to fix the stylus include:
A ring expected to be fixed, preferably glued, on the stylus;
means of axial and transverse positioning of the ring relative to the distal extremity of the arm, and
means keeping the ring on the distal extremity of the arm.

5. The system according to claim 4 wherein the axial and transverse positioning means include conical surface on the ring and a conical surface of a housing formed in the distal extremity, the aforesaid conical surfaces with substantially the same angle, and preferably expected to trap one in the other.

6. The system according to claim 4 wherein the means to maintain the ring includes on the aforesaid ring a peripheral rib and a nut, scheduled to be screwed onto the extremity and cooperating with the aforesaid rib to keep the ring gripping with the aforesaid extremity.

7. The system according to claim 1 wherein the stylus is one extremity of a flexible pipe.

8. The system according to claim 7 wherein the pipe is fully accessible and removable without tools.

9. The system according to claim 7 wherein the pipe has such a length that it provides an internal volume sufficient to store a sufficient sample volume for the inoculation.

10. The system according to claim 9 further comprising a roller to wind around a portion of the pipe.

11. The system according to claim 9 further comprising motorized pumping means, preferably with a syringe settled upstream of the pipe, expected for at least suck up and discharge the sample by the stylus.

12. The system according to claim 11 further comprising dispensing means settled so that the pumping means can also suck up at least one product in a corresponding reserve, and then discharge that aforesaid product by the stylus.

13. The system according to claim 12 wherein the dispensing means are arranged so that the pumping means can draw two products, independently of one another, each product having a specific reserve, and discharge independently each product by the stylus.

14. The system according to claim 13 wherein one of the products is a disinfectant, preferably an alcohol, and the other is a rinse aid, preferably distilled water.

15. The system according to claim 1 further comprising means to adapt a Petri dish wherein the Petri dish that is scheduled to contain the substrate.

16. A process for inoculating a Petri dish using a device according to claim 1.
